(19) United States
(12) Patent Application Publication
(10) Pub. No.: US 2018/0206758 A1
(43) Pub. Date: Jul. 26, 2018

(54) HAND HELD DEVICES FOR MAGNETIC INDUCTION TOMOGRAPHY

(71) Applicant: Kimberly-Clark Worldwide, Inc., Neenah, WI (US)

(72) Inventors: Joseph R. Feldkamp, Appleton, WI (US); Shawn Jeffery Sullivan, Neenah, WI (US); Andrew M. Long, Appleton, WI (US)

(21) Appl. No.: 15/747,562

(22) PCT Filed: Jul. 20, 2016
(86) PCT No.: PCT/US2016/043093

§ 371 Date: Jan. 25, 2018

Related U.S. Application Data
(60) Provisional application No. 62/209,933, filed on Aug. 26, 2015.

(51) Int. Cl. A61B 5/05; A61B 5/06; G01V 3/10

(52) U.S. Cl. CPC A61B 5/0522; A61B 5/061; A61B 2562/0223; A61B 2560/0425; G01V 3/10

(57) ABSTRACT

Apparatus, systems, and methods for magnetic induction tomography imaging using a hand held device are provided. More particularly, a magnetic induction tomography imaging system can include a hand held magnetic induction tomography device having a housing and at least one sensing unit. The housing of the hand held device can have a form factor such that the location of a hand holding the housing is separated from the sensing unit when the hand held device is in operation. The hand held magnetic imaging tomography device can include one or more electrical components separated in the housing (e.g., by shielding) from the at least one sensing unit to reduce electromagnetic interference between the at least one sensing unit and the one or more electrical components. A positioning system involving components included internally and/or externally to the hand held device can be used to determine position data for the hand held device.

### RELATED APPLICATIONS

The present application claims priority to U.S. provisional application Ser. No. 62/209,933 filed on Aug. 26, 2015, which is incorporated herein in its entirety by reference thereto.

### FIELD

The present disclosure relates generally to the field of magnetic induction tomography imaging, and more particularly to a hand held devices for magnetic induction tomography imaging.

### BACKGROUND

Magnetic induction tomography imaging can be used to image an electromagnetic property distribution (e.g. conductivity or permittivity) within tissues. More particularly, magnetic induction tomography techniques can provide for the low cost, contactless measurement of electromagnetic properties of tissue based on eddy currents induced in tissues by induction coils placed adjacent to the tissue.

Electromagnetic properties such as conductivity and permittivity vary spatially in tissue due to natural contrasts created by fat, bone, muscle and various organs. As a result, a conductivity or permittivity distribution obtained using magnetic induction tomography imaging techniques can be used to image various regions of the body, including lungs and abdominal regions, brain tissue, and other regions of the body that may or may not be difficult to image using other low cost biomedical imaging techniques, such as ultrasound. In this way, magnetic induction tomography imaging can be useful in the biomedical imaging of, for instance, wounds, ulcers, brain traumas, and other abnormal tissue states.

Existing techniques for magnetic induction tomography imaging typically involve the placement of a large number of coils (e.g. a coil array) near the sample and building an image based upon the measured mutual inductance of coil pairs within the large number of coils placed near the specimen. For instance, an array of source coils and an array of detection coils can be placed adjacent the specimen. One or more of the source coils can be energized using radiofrequency energy and a response can be measured at the detection coils. The conductivity distribution (or permittivity distribution) of the specimen can be determined from the response of the detection coils.

Magnetic induction tomography imaging can be conducted using measurements associated with a single coil. However, implementation of these techniques using a hand held device for collecting the coil measurements can pose several challenges. For instance, the hand of a technician using the device can create interference during scanning if the device is not held correctly. In addition, the power source, electronics, wires, and other elements can cause interference with the single coil, leading to less accurate coil measurements. In addition, for accurate magnetic induction tomography imaging, the position associated with each coil measurement is preferably known to a high degree of accuracy. This degree of precision can be difficult with hand held devices being physically moved from one location to another by a technician during scanning with the hand held device.

### SUMMARY

Aspects and advantages of embodiments of the present disclosure will be set forth in part in the following description, or may be learned from the description, or may be learned through practice of the embodiments.

One example aspect of the present disclosure is directed to a hand held magnetic induction tomography device. The hand held magnetic induction tomography device includes a housing and at least one sensing unit. Each sensing unit includes a single coil. The hand held magnetic induction tomography device is configured to obtain a coil measurement with the sensing unit when the single coil is placed adjacent to a specimen. The system further includes a positioning system configured to determine a position of the hand held magnetic induction tomography device associated with each coil measurement. The system further includes a map generation system configured to generate an electromagnetic property map of at least a portion of the specimen based at least in part on the coil measurement.

Another example aspect of the present disclosure is directed to a hand held magnetic induction tomography device. The hand held magnetic induction tomography device can include a housing having a form factor to facilitate holding by hand and at least one sensing unit. Each sensing unit includes a single coil. The hand held magnetic induction tomography device further includes one or more electrical components separated from the at least one sensing unit a sufficient distance to reduce electromagnetic interference between the one or more electrical components and the at least one sensing unit. The hand held magnetic induction tomography device can be configured to obtain a coil measurement with the sensing unit when the single coil is placed adjacent to a specimen.

Yet another example aspect of the present disclosure is directed to a method for magnetic induction tomography imaging. The method includes accessing a plurality of coil property measurements obtained for a specimen using a single coil of a hand held magnetic induction tomography device. Each of the coil property measurements can be obtained with the single coil at one of a plurality of discrete locations relative to the specimen. The method includes associating coil position data with each of the plurality of coil property measurements. The coil position data can be indicative of the position and orientation of the single coil relative to the specimen for each coil measurement. The coil position can be obtained using a positioning system configured to determine a position of the hand held magnetic induction tomography device. The method further includes accessing a model defining a relationship between coil property measurements obtained by the single coil and an electromagnetic property of the specimen and generating a three-dimensional electromagnetic property map of the specimen using the model based at least in part on the plurality of coil property measurements and the coil position data associated with each coil measurement.

Variations and modifications can be made to these example aspects of the present disclosure.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussions of embodiments directed to one of ordinary skill in the art are set forth in the specification, which makes reference to the appended figures, in which:

FIG. 1 depicts an example system for magnetic induction tomography imaging using a hand held device according to example embodiments of the present disclosure;

FIG. 2 depicts a perspective view of an example hand held device according to example embodiments of the present disclosure;

FIG. 3 depicts a side view of an example hand held device according to example embodiments of the present disclosure;

FIGS. 4-5 depict example conductivity maps generated according to example embodiments of the present disclosure;

FIG. 6 depicts an example coil for magnetic induction tomography imaging according to example embodiments of the present disclosure;

FIG. 7 depicts example connection traces for a coil for magnetic induction tomography imaging according to example embodiments of the present disclosure;

FIG. 8 depicts a process flow diagram of an example method for providing a coil for magnetic induction tomography imaging according to example embodiments of the present disclosure;

FIG. 9 depicts a block diagram of an example circuit associated with a coil used for magnetic induction tomography imaging according to example embodiments of the present disclosure; and

FIG. 10 depicts a process flow diagram of an example method for magnetic induction tomography imaging according to example embodiments of the present disclosure.

### DETAILED DESCRIPTION

Reference now will be made in detail to embodiments, one or more examples of which are illustrated in the drawings. Each example is provided by way of explanation of the embodiments, not limitation of the invention. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

### Overview

Generally, example aspects of the present disclosure are directed to hand held devices for magnetic induction tomography imaging of a specimen, such as a tissue specimen, using measurements associated with a single coil. More particularly, a plurality of coil property measurements can be obtained using a single coil at a plurality of different discrete locations relative to the specimen using a hand held device. A three-dimensional electromagnetic property map, such as a three-dimensional conductivity map or a three-dimensional permittivity map, can be generated from the plurality of coil property measurements. In this way, a simple and cost effective way of imaging tissue can be provided using contactless coil property measurements obtained using a hand held device.

More particularly, a magnetic induction tomography imaging system can include a hand held magnetic induction tomography device having a housing and at least one sensing unit. The at least one sensing unit can include a single coil. In some embodiments, the housing can have a form factor to facilitate holding of the hand held device by hand, such as a technician's hand. For instance, the housing can have a size, shape, and geometry to facilitate holding of the hand held device by hand. Providing a portable hand held device for magnetic induction tomography imaging can increase the ease and flexibility of performing coil measurements of a specimen in a magnetic induction tomography imaging system.

In some embodiments, the housing of the hand held device can have a form factor such that the location of a hand grasping or otherwise holding the housing is separated (e.g., separated a threshold distance) from the sensing unit when the hand held device is in operation. For instance, a grip portion of the hand held device can be located a threshold distance away from the sensing unit. In this way, interference resulting from placement of a technician's hand near the single coil of the at least one sensing unit during acquisition of coil measurements can be reduced.

The housing of the hand held device can accommodate at least one sensing unit having a single coil. In some embodiments, the coil can include a plurality of concentric conductive circular loops with spacing sufficient between the loops, or sufficiently different radii, to reduce capacitive coupling with the specimen. The conductive loops can be connected in series with connection traces without allowing the connection traces to distort the fields produced by the plurality of concentric conductive circular loops. The plurality of concentric conductive loops can be arranged in multiple planes (e.g., on a multilayer printed circuit board) as a two layer stack. The spacing between the planes or the plane separation distance can be selected such that mathematically the plurality of conductive loops can be treated as being located in a common plane for purposes of a quantitative analytical model. For instance, the plane separation distance can be in the range of about 0.2 mm to about 0.7 mm, such as about 0.5 mm. As used herein, the use of the term “about” with reference to a dimension or other characteristic is intended to refer to within 30% of the specified dimension or other characteristic.

In some embodiments, the hand held magnetic induction tomography device can include a housing that can accommodate different sized sensing units. For instance, the housing can accommodate modular sensing units (e.g., cartridges) that can be interchanged with one another on the hand held device (e.g., using Velcro fasteners or other suitable fasteners or attachment mechanisms to facilitate rapid interchangeability of the sensing units). Each sensing unit can have a coil with different coil dimensions relative to the other sensing units to provide for different depths of measurement by the hand held magnetic induction tomography device. In some embodiments, the hand held magnetic induction tomography device can accommodate a plurality of sensing units. Each sensing unit can include a single coil for performing a coil measurement. In particular implementations, each of the plurality of sensing units can include coils with different coil dimensions so that the hand held magnetic induction tomography device can support measurements at different depths without having to interchange sensing units on the hand held magnetic induction tomography device.

In some embodiments, the hand held magnetic imaging tomography device can include one or more electrical and/or mechanical components that can be used to support operation of the hand held magnetic imaging tomography device. For instance, the hand held device can include one or more electrical components such as an a power source (e.g., one or more batteries), RF energy source (e.g., an oscillator circuit), a measurement circuit used to drive the sensing unit and obtain coil measurements, one or more processors (e.g., microcontrollers) used to control various aspects of the hand held device, one or more memory devices to store coil measurements, one or more positioning devices (e.g., optical, electromagnetic, or other motion sensors used to determine position and/or orientation of the hand held device), and one or more communication devices.

In some embodiments, the one or more electrical components can be disposed within the housing of the hand held device, such as on one or more printed circuit boards within the housing of the hand held device. The one or more electrical and/or mechanical components can be separated in the housing from the at least one sensing unit to reduce electromagnetic interference between the at least one sensing unit and the one or more electrical and/or mechanical components. In particular implementations, the hand held device can include shielding used to separate the at least one sensing unit from the one or more electrical and/or mechanical components of the hand held device.

In some embodiments, the one or more electrical and/or mechanical components used to support operation of the hand held device can be located at a remote station. For instance, one or more of the electrical components described above can be located at a remote station to reduce interference with the at least one sensing unit. The hand held device can communicate with the one or more electrical components located at the remote station using a suitable communications interface, such as any suitable wired or wireless communication interface or combination thereof. In particular implementations, the remote station can be located on a movable cart or other movable apparatus to facilitate placement of the remote station near the hand held device when the hand held device is performing measurements of the specimen.

According to particular aspects of the present disclosure, the magnetic induction tomography system can further include a positioning system configured to obtain positioning data for each coil measurement performed by the hand held device. The positioning system can be configured to determine data indicative of a position and/or orientation for each coil measurement for use in generating an electromagnetic property map of a specimen.

In one embodiment, the positioning system can include an optical positioning system. The optical positioning system can use one or more of infrared sensors, lasers, and/or one or more cameras or other image capture devices to determine the position of the hand held device when performing a coil measurement. For instance, in one implementation, the positioning system includes at least one camera configured to capture an image of the hand held device during the performance of the measurement. The image can be processed to identify the location of the hand held device in the image. For instance, pattern recognition techniques can be used to determine the position of the hand held device in the image based on a pattern or reflective element located on the hand held device. Based on the position of the hand held device in the image, the positioning system can calculate the position and/or orientation of the hand held device and the position and/or orientation of the single coil performing the coil measurement for use in generating an electromagnetic property map of the specimen.

In some embodiments, the positioning system can include an electromagnetic positioning system. For instance, the positioning system can include a low frequency (Pohemus) positioning system and/or a radar (UHF) positioning system. In some embodiments, the positioning system can include an acoustical positioning system, such as a sonar positioning system. In still other embodiments, signals from one or more sensors on the hand held device itself (e.g., motion sensors, inertia sensors, lasers, depth sensors, cameras, etc.) can be used to determine the position and/or orientation of the hand held device relative to the specimen.

The system can further include a map generation system configured to generate an electromagnetic property map (e.g., a conductivity map) of at least a portion of the specimen based at least in part on the coil property measurement. The map generation system can be located on the hand held device or located at a remote station in communication with the hand held device.

According to particular embodiments, the magnetic induction tomography imaging can be performed based at least in part on a model that defines a relationship between coil measurements and an electromagnetic property distribution of a specimen. In one implementation, the model is a quantitative analytical model that describes the real part of a change in impedance (e.g., ohmic loss) of a single planar multi-loop coil, having a plurality of concentric conductive loops, resulting from induced eddy currents when excited with RF energy and placed near to arbitrarily shaped objects with arbitrary three-dimensional conductivity distributions.

Using the model, a three-dimensional electromagnetic property map can be generated for tissue using the plurality of coil property measurements. For instance, a plurality of coil loss measurements obtained for the specimen can be accessed. Each coil property measurement can be associated with one of a plurality of discrete locations relative to the specimen. Position data can be associated with each coil property measurement. The position data can be indicative of the position and orientation of the single coil when the measurement was performed.

Once a plurality of coil property measurements and associated position data have been obtained, inversion of the obtained coil property measurements can be performed using the model to obtain a three-dimensional electromagnetic property map indicative of the electromagnetic property distribution (e.g. conductivity distribution) of the specimen leading to the plurality of obtained measurements. In one particular implementation, the inversion can be performed by discretizing the specimen into a finite element mesh. A non-linear or constrained least squares solver can determine an electromagnetic property distribution for the finite element mesh that most likely contributes to the plurality of obtained coil property measurements. The solved conductivity distribution can be output as the three-dimensional conductivity map for the specimen.

### Example Systems for Magnetic Induction Tomography Imaging

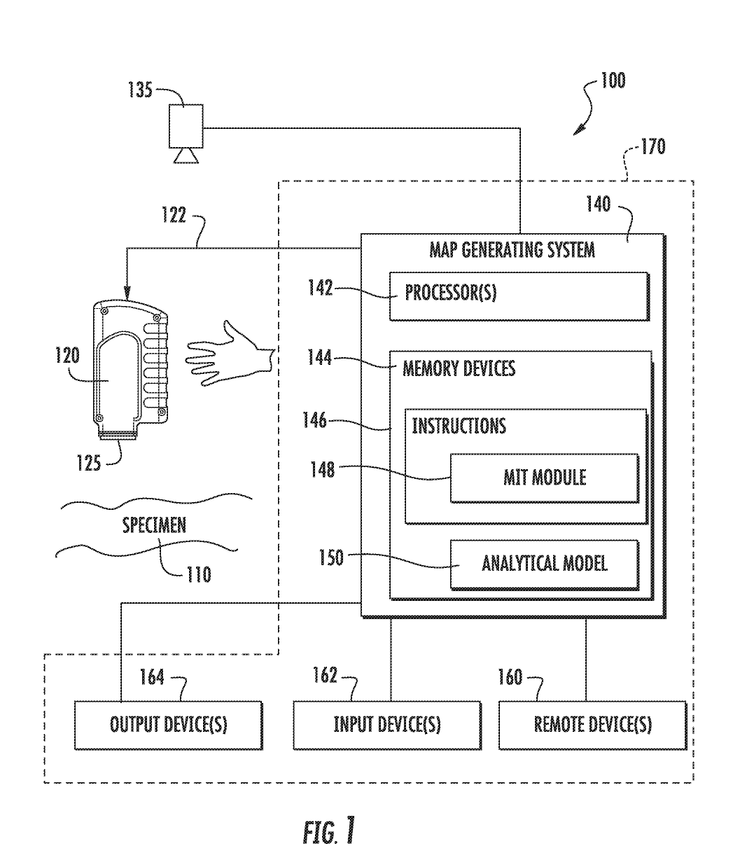

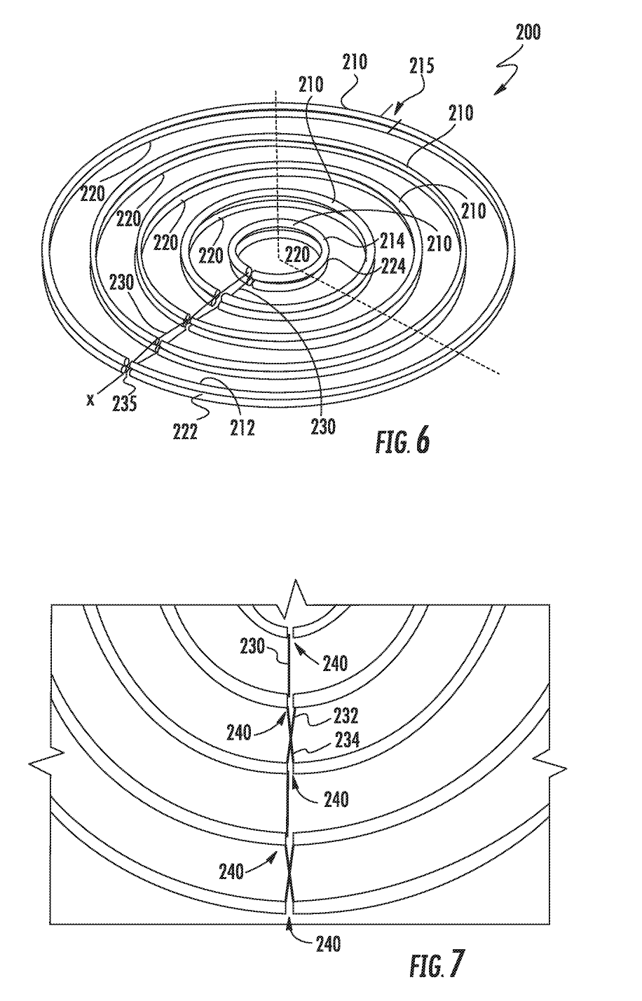

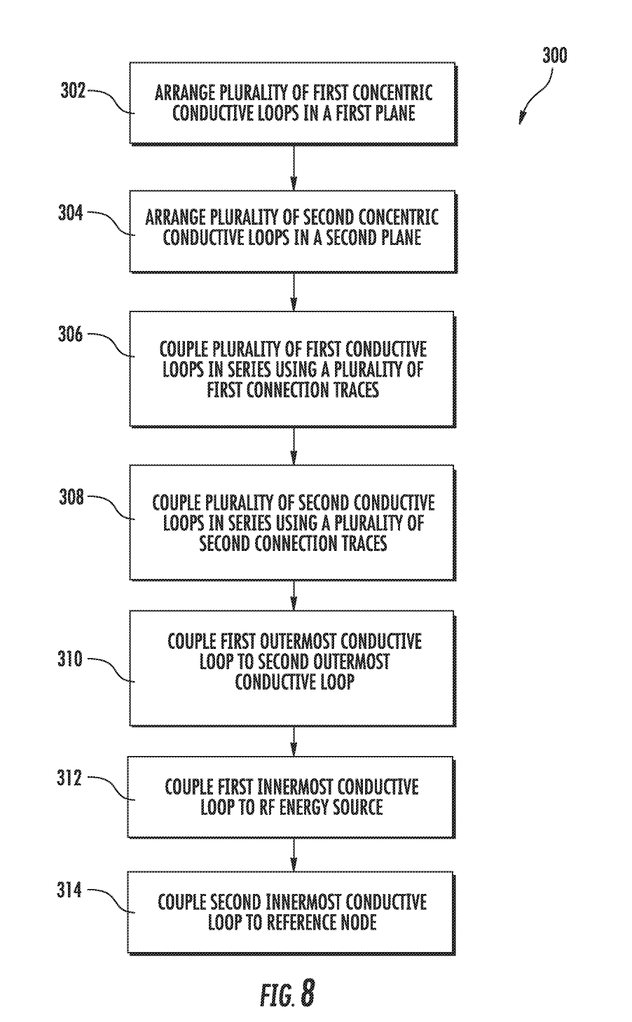

FIG. 1 depicts an example system 100 for magnetic induction tomography imaging of a specimen 110, such as a human tissue or animal tissue specimen. The system 100 includes a hand held device 120 having at least one sensing unit 125 for obtaining coil property measurements for magnetic induction tomography imaging according to example aspects of the present disclosure. The sensing unit 125 can include a single coil having a plurality of concentric conductive loops disposed in one or more planes on a printed circuit board. One example coil design for magnetic induction tomography imaging according to example aspects of the present disclosure will be discussed in more detail below with reference to FIGS. 6 and 7 below.

Example aspects of the present disclosure will be discussed with reference to a hand held device 120 having one sensing unit for purposes of illustration and discussion. Those of ordinary skill in the art, using the disclosures provided herein, will understand that the hand held device 120 can include a plurality of sensing units. Each sensing unit can include a single coil. Independent measurements associated with each single coil can be used to generate an electromagnetic property map as will be discussed in more detail below without dependence on measurements from coils associated with other sensing units.

The hand held device 120 of FIG. 1 can include an RF energy source (e.g., an oscillator circuit) configured to energize the coil of sensing unit 125 with RF energy at an excitation frequency (e.g. 12.5 MHz) when the sensing unit 125 is placed adjacent to the specimen 110. The energized coil of the sensing unit 125 can generate magnetic fields, which can induce eddy currents in the specimen 110. These induced eddy currents in the specimen can cause a coil loss (e.g. a change in impedance) of the coil of the sensing unit 125. The hand held device 120 can include circuitry and electrical components (e.g., a measurement circuit) for determining the coil loss associated with the coil of the sensing unit 125 during a coil property measurement at a particular location relative to the specimen 110.

Coil property measurements can be obtained using the single coil of the sensing unit 125 while the hand held device 120 is positioned at a variety of different locations and orientations relative to the specimen 110. The collected coil property measurements can be provided to a map generation system 140 (e.g., a computing system programmed to generate electromagnetic property maps from coil measurements) where the coil property measurements can be analyzed to generate a three-dimensional electromagnetic property map of the specimen 110, such as a three-dimensional conductivity map or a three-dimensional permittivity map of the specimen 110.

According to particular aspects of the present disclosure, the hand held device 120 can be manually positioned at a plurality of discrete locations for performance of the coil property measurement. For instance, a medical professional can manually position a hand held coil device 120 relative to the specimen 110 to obtain coil property measurements at a plurality of discrete locations relative to the specimen 110.

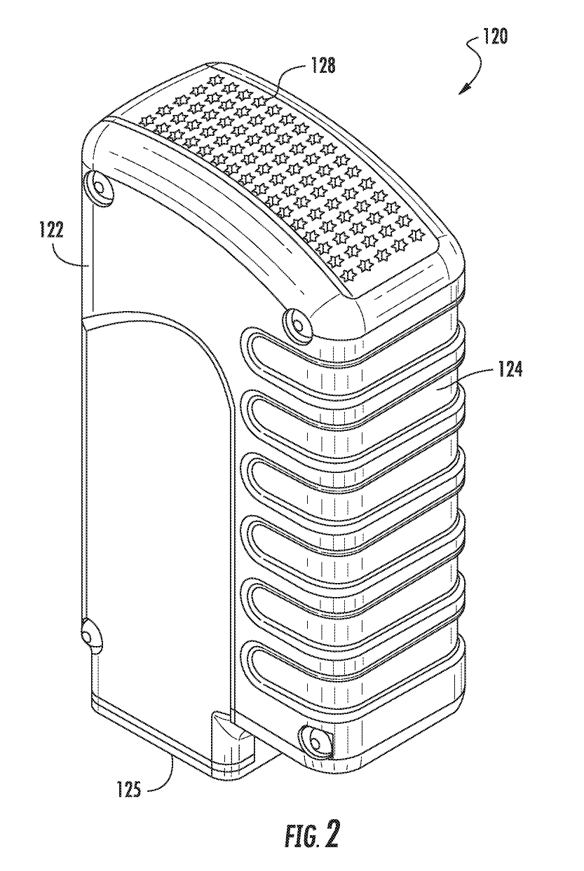

FIG. 2 depicts a perspective view on one example embodiment of a hand held device 120 for magnetic induction tomography imaging according to example embodiments of the present disclosure. As shown, the hand held device 120 includes a housing 122 for storing and protecting various components (e.g., electrical components) of the hand held device 120 used to support acquisition of coil measurements using sensing unit 125.

The example hand held device 120 of FIG. 2 includes a form factor to facilitate holding the hand held device 120 by hand during acquisition of coil measurements. For instance, the hand held device 120 includes a grip portion 124. As illustrated in FIG. 2, the grip portion 124 can include one or more grooves or channels to facilitate grasping or holding the hand held device by hand 120. The hand held device 120 further includes a form factor such that the location of a hand grasping the housing when in operation is separated a threshold distance from the single coil of the sensing unit 125. For instance, the grip portion 124 can be located in the range of about 0.5 inches to about 6 inches away from the sensing unit 125, such as about 2 inches to 4 inches away from the sensing unit, such as about 3 inches away from the sensing unit. In this way, interference between a technician's hand and the sensing unit 125 can be reduced while performing measurements with the hand held device 120.

The hand held device 120 depicts one example form factor according to example embodiments of the present disclosure to facilitate holding the device by hand. Those of ordinary skill in the art, using the disclosures provided herein, should understand that other form factors are contemplated. For instance, the hand held device 120 can have a housing having a first portion that has a first shape adapted to conform to the sensing unit 125 and a second portion that is a different shape (e.g., a cylindrical shape) that is adapted to be held by hand during operation.

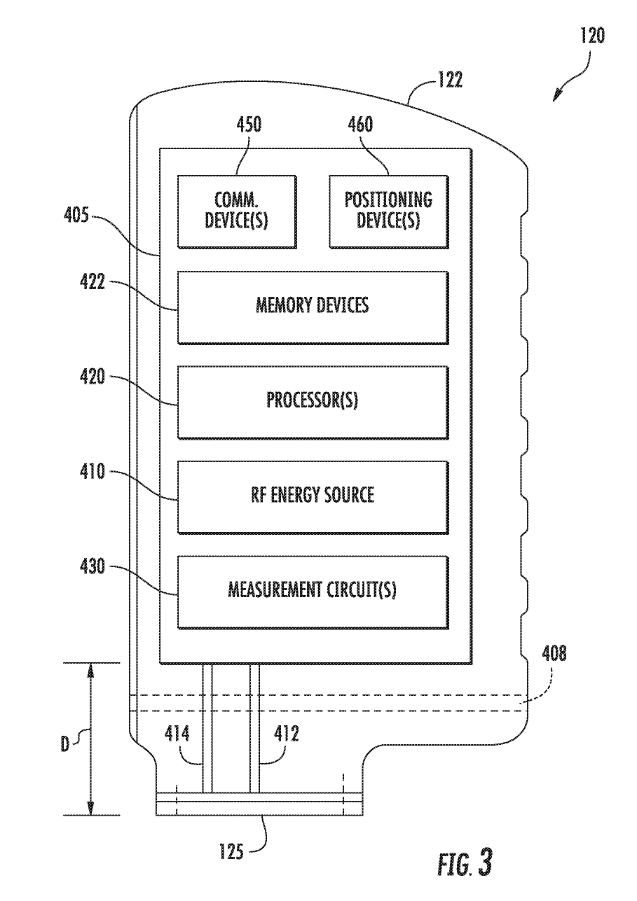

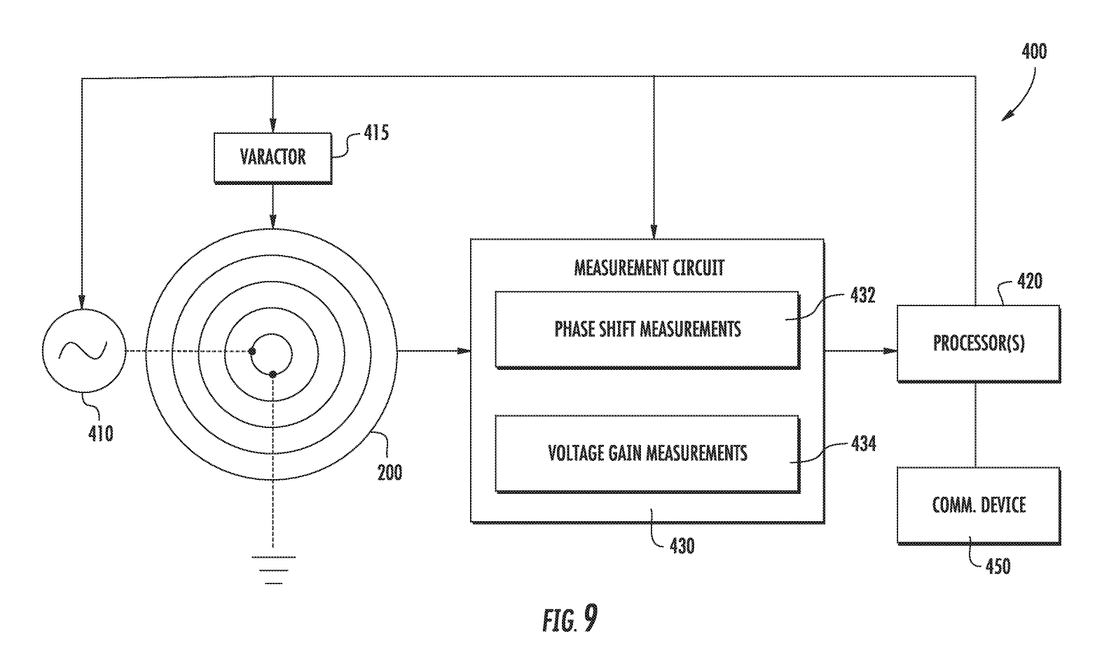

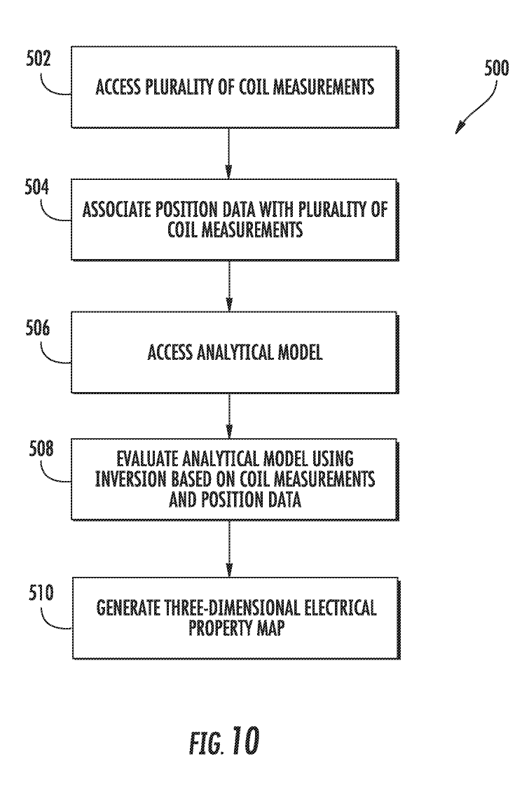

As shown in FIG. 3, the hand held device 120 can include one or more electrical components to support operation of the hand held device 120. The one or more electrical components can include a power source such as a battery (not shown), an RF energy source 410, processor(s) 420, memory device(s) 422, measurement circuit(s) 430, communication device(s) 450, and positioning device(s) 460. Operation of selected of the above electrical components will be discussed in more detail with reference to FIG. 9 below.

Referring to FIG. 3, the RF energy source 410 (e.g., an oscillator circuit) can be configured to generate RF energy for energizing the coil of the sensing unit 125. The processor(s) 420 can be configured to control various aspects of the circuit 400 as well as to process information obtained by the circuit 400 (e.g., information obtained by measurement circuit 430). The processor(s) 420 can include any suitable processing device, such as digital signal processor, microprocessor, microcontroller, integrated circuit or other suitable processing device. The memory devices 422 can be configured to store information and data collected by the hand held device 120. For instance, the memory devices 422 can be configured to store coil measurements obtained by the sensing unit 125. The memory devices 422 can include single or multiple portions of one or more varieties of tangible, non-transitory computer-readable media, including, but not limited to, RAM, ROM, hard drives, flash drives, optical media, magnetic media or other memory devices. The measurement circuit 430 can be configured to obtain coil measurements of the single coil of the sensing unit 125. Details of an example measurement circuit are discussed with reference to FIG. 9 below.

The positioning device(s) 460 of FIG. 3 can include circuitry for supporting one or more sensors used to determine the position and/or orientation of the hand held device 120 when performing coil measurements. For instance, the positioning device(s) 460 can include motion sensors (e.g., accelerometers, compass, magnetometers, gyroscopes, etc.) and other suitable sensors that provide signals indicative of the orientation of the hand held device 120. Further, the hand held device 120 can include depth sensors (e.g., laser sensors, infrared sensors, image capture devices) that can be used to determine a depth or distance of the hand held device 120 to a specimen. Signals from the positioning device(s) 460 can be used in determining a position and/or orientation associated with each coil measurement.

The communication device(s) 450 can be used to communicate information from the hand held device 120 to a remote location, such as a remote computing device. The communication device(s) can include, for instance, transmitters, receivers, ports, controllers, antennas, or other suitable components for communicating information from the hand held device 120 over a wired and/or wireless network.

The various electrical components supporting operation of the hand held device 120 can be disposed on a printed circuit board 405 within the housing 122 of the hand held device 120. As illustrated, in FIG. 3, the one or more electrical components can be separated a threshold distance D from the sensing unit 125 so as to reduce interference between the one or more electrical components and the sensing unit 125. In particular embodiments, the threshold distance D can be in the range of about 0.5 inches to about 4 inches, such as about 2 inches to 3 inches away, such as about 2 inches.

As shown in FIG. 3, the hand held device 120 can further include a shield 408. The shield 408 can be manufactured from a conductive material or high dielectric constant, non-lossy material. The shield 408 can separate the sensing unit 125 from the electrical components supporting operation of the hand held device 120 to further reduce electromagnetic interference between the electrical components and the sensing unit 125. Conductive paths 412 and 414 passing through the shield 408 can be used to communicate signals from the sensing unit 125 to the electrical components supporting operation of the hand held device 120.

One or more of the electrical components supporting operation of the hand held device and other components of the magnetic induction tomography system can be located at a location remote from the hand held device 120. For instance, as shown in FIG. 1, a map generation system 140 is located remote from the hand held device 120. The map generation system 140 can be configured to generate one or more electromagnetic property maps based on measurements obtained by the hand held device 120 as will be discussed in more detail below. The map generation system 120 can be located on a movable cart 170 or other device to make the map generation system 120 portable. The hand held device 120 can be configured to communicate with the map generation system 140 over a communication interface 122. The communication interface 122 can be any suitable wired or wireless interface or combination of wired and wireless links.

To generate an accurate three-dimensional electromagnetic property map of the specimen 110, position data needs to be associated with the coil property measurements obtained by the hand held device 120. The position data can be indicative of the position (e.g., as defined by an x-axis, y-axis, and a z-axis relative to the specimen 110) of the coil 125 as well as an orientation of the coil 125 (e.g., tilt angle(s) relative to the specimen 110). The magnetic induction tomography system 100 according to example embodiments of the present disclosure includes a positioning system to determine the position data associated with measurements obtained by the hand held device 120.

One example positioning system according to aspects of the present disclosure includes an optical positioning system. For instance, the positioning system can include at least one camera 135 positioned above the specimen 110. The camera 135 can be configured to capture images of the hand held device 120 as the hand held device 120 obtains measurements of the specimen 110. The camera can capture images at a variety of wavelengths or spectrums, including one or more wavelengths in the ultraviolet, infrared or visible spectrum.

Images captured by the camera 135 can be processed to determine the position of the hand held device 120 and the sensing unit 125. In some embodiments, the hand held device 120 can also include a graphic located on a surface of the coil device 120. One example graphic 128 is depicted in FIG. 2. As the plurality of coil property measurements are performed, the image capture device 135 can capture images of the graphic 128. The images can be processed to determine the position of the hand held device 120 based on the position of the graphic in the images. In particular implementations, the camera 135 can include a telecentric lens to reduce error resulting from parallax effects. Other suitable optical positioning systems can be used to determine the position of the hand held device 120, such as infrared based systems, laser based systems, or other suitable systems.

For instance, in one embodiment, the hand held device 120 includes reflective markers that are attached to the outside of the hand held device 120. The reflective markers can be configured to reflect visible light, ultraviolet light, infrared light, or other suitable light. The hand held device 120 can have a form factor such that the reflective markers are maintained within light of sight of the camera 135 during operation. For instance, the reflective markers can be located on a surface opposite the sensing unit 125 so that the reflective markers are within the line of sight of the camera 135 when performing measurements with the hand held device 120. In one embodiment, the reflective markers are disposed on an axis that parallels an axis associated with the sensing unit 125. The reflective markers can be disposed on a surface of the hand held device 120 that is the greatest distance from the sensing unit 125.

The camera 135 can capture images of the hand held device 120. The positioning system can determine the location of the hand held device based at least in part on the location of the reflective markers in the images captured of the hand held device 120 by the camera 135.

In some embodiments, the positioning system can include an electromagnetic positioning system. For instance, the positioning system can include a low frequency (Pohemus) positioning system and/or a radar (UHF) positioning system. In some embodiments, the positioning system can include an acoustical positioning system, such as a sonar positioning system.

In some embodiments, the hand held device 120 can include one or more motion sensors (e.g., a three-axis accelerometer, gyroscope, and/or other motion sensors) and/or one or more depth sensors. The orientation of the single coil 125 relative to the surface can be determined using the signals from the motion sensor(s). For instance, signals from a three-axis accelerometer can be used to determine the orientation of the sensing unit 125 during a coil property measurement. The depth sensor(s) can be used to determine the distance from the single coil to the specimen 110 (e.g., the position along the z-axis). The depth sensor(s) can include one or more devices configured to determine the location of the sensing unit 125 relative to a surface. For instance, the depth sensor(s) can include one or more laser sensor devices and/or acoustic location sensors. In another implementation, the depth sensor(s) can include one or more cameras configured to capture images of the specimen 110. The images can be processed to determine depth to the specimen 110 using, for instance, structure-from-motion techniques.

The map generation system 140 can receive the coil property measurements, together with coil location and orientation data, and can process the data to generate a three-dimensional electromagnetic property map of the specimen 110. The map generation system 140 is depicted as being located remotely from the hand held device 120 in FIG. 1. However, in other embodiments, the map generation system 140 can be included as part of the hand held device 120.

The map generation system 140 can include one or more computing devices, such as one or more of a desktop, laptop, server, mobile device, display with one or more processors, or other suitable computing device having one or more processors and one or more memory devices. The map generation system 140 can be implemented using one or more networked computers (e.g., in a cluster or other distributed computing system). For instance, the map generation system 140 can be in communication with one or more remote devices 160 (e.g., over a wired or wireless connection or network).

The computing system 140 includes one or more processors 142 and one or more memory devices 144. The one or more processors 142 can include any suitable processing device, such as a microprocessor, microcontroller, integrated circuit or other suitable processing device. The memory devices 144 can include single or multiple portions of one or more varieties of tangible, non-transitory computer-readable media, including, but not limited to, RAM, ROM, hard drives, flash drives, optical media, magnetic media or other memory devices. The map generation system 140 can further include one or more input devices 162 (e.g., keyboard, mouse, touchscreen, touchpad, microphone, etc.) and one or more output devices 164 (e.g. display, speakers, etc.).

The memory devices 144 can store instructions 146 that when executed by the one or more processors 142 cause the one or more processors 142 to perform operations. The map generation system 140 can be adapted to function as a special-purpose machine providing desired functionality by accessing the instructions 146. The instructions 146 can be implemented in hardware or in software. When software is used, any suitable programming, scripting, or other type of language or combinations of languages may be used to implement the teachings contained herein.

As illustrated, the memory devices 144 can store instructions 146 that when executed by the one or more processors 142 cause the one or more processors 142 to implement a magnetic induction tomography (“MIT”) module 148. The MIT module 148 can be configured to implement one or more of the methods disclosed herein for magnetic induction tomography imaging using a single coil, such as the method disclosed in FIG. 10.

The one or more memory devices 144 of FIG. 1 can also store data, such as coil property measurements, position data, three-dimensional electromagnetic property maps, and other data. As shown, the one or more memory devices 144 can store data associated with an analytical model 150. The analytical model 150 can define a relationship between coil property measurements obtained by a single coil and an electromagnetic property distribution of the specimen 110. Features of an example analytical model will be discussed in more detail below.

MIT module 148 may be configured to receive input data from input device 162, from coil device 120, from the positioning system, from data that is stored in the one or more memory devices 144, or other sources. The MIT module 148 can then analyze such data in accordance with the disclosed methods, and provide useable output such as three-dimensional electromagnetic property maps to a user via output device 164. Analysis may alternatively be implemented by one or more remote device(s) 160.

The technology discussed herein makes reference to computing systems, servers, databases, software applications, and other computer-based systems, as well as actions taken and information sent to and from such systems. One of ordinary skill in the art, using the disclosures provided herein, will recognize that the inherent flexibility of computer-based systems allows for a great variety of possible configurations, combinations, and divisions of tasks and functionality between and among components. For instance, processes discussed herein may be implemented using a single computing device or multiple computing devices working in combination. Databases and applications may be implemented on a single system or distributed across multiple systems. Distributed components may operate sequentially or in parallel.

FIG. 4 depicts one example conductivity map 180 that can be generated by the system 100 from a plurality of coil property measurements using a hand held device according to an example embodiment of the present disclosure. The conductivity map 180 can provide a two-dimensional view of a cross-section of a three-dimensional conductivity map generated by the MIT module 148 of FIG. 1 based on measurements obtained by the hand held device 120. The conductivity map 180 of FIG. 4 can be presented, for instance, on the output device 164 of the computing system 140 of FIG. 1.

The conductivity map 180 of FIG. 4 provides a transverse view of a spinal column of a patient, transecting and revealing the spinal canal. The conductivity map 180 plots conductivity distribution along x-, y-, and z-axes in units of centimeters. The conductivity map 180 includes a scale 182 indicative of grey scale colors associated with varying degrees of conductivity in units of S/m. As shown, the conductivity map 180 shows the contrasting conductivity of regions of human tissue in the spinal region and can provide an image of the spinal region of the patient.

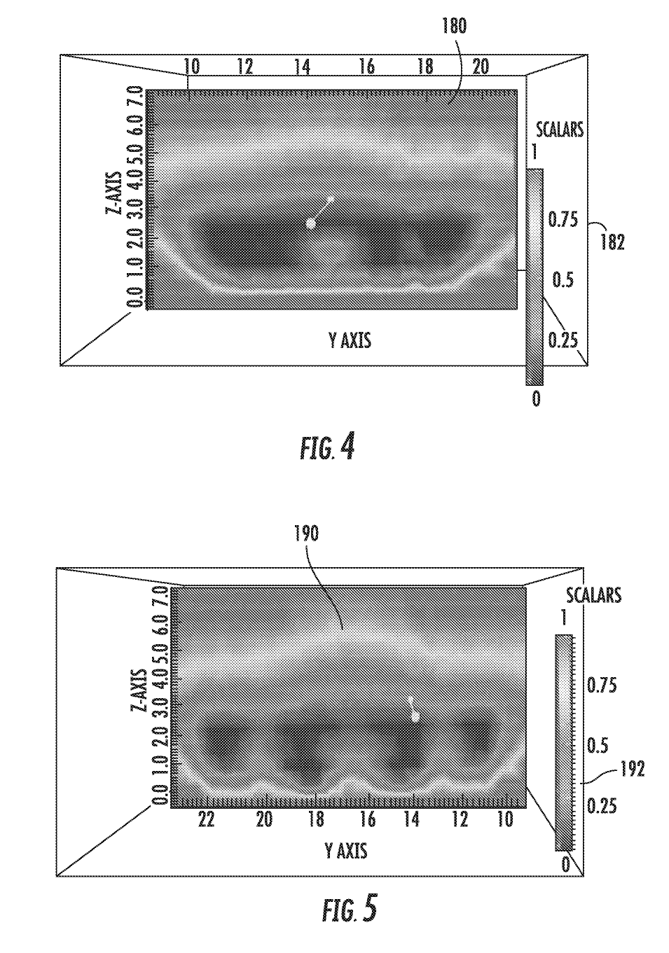

FIG. 5 depicts another example conductivity map 190 that can be generated by the system 100 from a plurality of coil property measurements using a single coil according to example embodiments of the present disclosure. The conductivity map 190 can be a two-dimensional view of a cross-section of a three-dimensional conductivity map generated by the MIT module 148 of FIG. 1 based on measurements obtained by the hand held device 120. The conductivity map 190 of FIG. 5 can be presented, for instance, on the output device 164 of the computing system 140 of FIG. 1.

The conductivity map 190 of FIG. 5 provides a sagittal view of the spinal region of a patient, offset but parallel to the spinal column. The conductivity map 190 plots conductivity distribution along x-, y-, and z-axes in units of centimeters. The conductivity map 190 includes a scale 192 indicative of grey scale colors associated with varying degrees of conductivity, in units of S/m. As shown, the conductivity map 190 shows the contrasting conductivity of regions of human tissue in the spinal region and can provide an image of the spinal region of the patient. This slice transects and reveals the structure associated with the connection of ribs to transverse processes of the vertebrae. The conductivity map 180 and the conductivity map 190, together with other views, can provide varying images of the spinal region of the patient for diagnostic and other purposes.

### Example Quantitative Analytical Model for a Single Coil

An example quantitative analytical model for obtaining a three-dimensional conductivity map from a plurality of coil property measurements obtained by a hand held device will now be set forth. The quantitative model is developed for an arbitrary conductivity distribution, but with permittivity and magnetic permeability treated as spatially uniform. The quantitative analytical model was developed for a coil geometry that includes a plurality of concentric circular loops, all lying within a common plane and connected in series, with the transient current considered to have the same value at all points along the loops. A conductivity distribution is permitted to vary arbitrarily in space while a solution for the electric field is pursued with a limit of small conductivity (<10 S/m). Charge free conditions are assumed to hold, whereby the electrical field is considered to have zero divergence. Under these conditions, fields are due only to external and eddy currents.

The quantitative analytical model can correlate a change in the real part of impedance (e.g., ohmic loss) of the coil with various parameters, including the conductivity distribution of the specimen, the position and orientation of the single coil relative to the specimen, coil geometry (e.g. the radius of each of the plurality of concentric conductive loops) and other parameters. One example model is provided below:

$$- {δZ}_{re} = \frac{{μ}^{2} {ω}^{2}}{4 {π}^{2}} {\sum }_{j , k} \sqrt{{ρ}_{j} {ρ}_{k}} \int {d}^{3} x \frac{\breve{σ} ( \vec{r} )}{ρ} {Q}_{\frac{1}{2}} ( {η}_{j} ) {Q}_{\frac{1}{2}} ( {η}_{k} )$$

−δZᵣₑ is the coil property measurement (e.g., the real part of the impedance loss of the coil). μ is the magnetic permeability in free space. ω is the excitation frequency of the coil. ρₖ and ρⱼ are the radii of each conductive loop j and k for each interacting loop pair j,k. The function Q_{1/2} is known as a ring function or toroidal harmonic function, which has the argument ηⱼ and ηₖ as shown here:

$${η}_{j} = \frac{{ρ}^{2} + {ρ}_{j}^{2} + {z}^{2}}{2 {ρρ}_{j}}$$

$${n}_{k} = \frac{{ρ}^{2} + {ρ}_{k}^{2} + {z}^{2}}{2 {ρρ}_{k}}$$

With reference to a coordinate system placed at the center of the concentric loops, such that loops all lie within the XY-plane, ρ measures radial distance from coil axis to a point within the specimen while z measures distance from the coil plane to the same point within the specimen.

The model introduces electrical conductivity \check{σ}(\vec{P}) as a function of position. The integrals can be evaluated using a finite element mesh (e.g., with tetrahedral elements) to generate the conductivity distribution for a plurality of coil property measurements as will be discussed in more detail below.

### Example Coil Designs for Magnetic Induction Tomography Imaging

An example coil design that approximates the coil contemplated by the example quantitative model will now be set forth. A coil according to example aspects of the present disclosure can include a plurality of concentric conductive loops arranged in two-planes on a multilayer printed circuit board. The plurality of concentric conductive loops can include a plurality of first concentric conductive loops located within a first plane and a plurality of second concentric conductive loops located in a second plane. The second plane can be spaced apart from the first plane by a plane separation distance. The plane separation distance can be selected such that the coil approximates the single plane coil contemplated in the example quantitative analytical model for magnetic induction tomography imaging disclosed herein.

In addition, the plurality of conductive loops can be connected in series using a plurality of connection traces. The plurality of connection traces can be arranged so that the contribution to the fields generated by the connection traces can be reduced. In this manner, the coil according to example aspects of the present disclosure can exhibit behavior that approximates a plurality of circular loops arranged concentric to one another and located in the same plane.

FIG. 6 depicts an example coil 200 used for magnetic induction tomography imaging according to example aspects of the present disclosure. As shown, the coil 200 includes ten concentric conductive loops. More particularly, the coil 200 includes five first concentric conductive loops 210 disposed in a first plane and five second concentric conductive loops 220 disposed in a second plane. The first and second concentric conductive loops 210 and 220 can be 1 mm or 0.5 mm copper traces on a multilayer printed circuit board. In one example implementation, the radii of the five concentric conductive loops in either plane are set at about 4 mm, 8 mm, 12 mm, 16 mm, and 20 mm respectively. Other suitable dimensions and spacing can be used without deviating from the scope of the present disclosure.

As shown, each of the plurality of first concentric conductive loops 210 is disposed such that it overlaps one of the plurality of second concentric conductive loops 220. In addition, the first concentric conductive loops 210 and the second concentric conductive loops 220 can be separated by a plane separation distance. The plane separation distance can be selected such that the coil 200 approximates a single plane of concentric loops as contemplated by the quantitative analytical model. For instance, the plane separation distance can be in the range of about 0.2 mm to about 0.7 mm, such as about 0.5 mm.

The plurality of first conductive loops 210 can include a first innermost conductive loop 214. The first innermost conductive loop 214 can be coupled to an RF energy source. The plurality of second conductive loops 220 can include a second innermost conductive loop 224. The second innermost conductive loop 224 can be coupled to a reference node (e.g. a ground node or common node).

The coil further includes a plurality of connection traces 230 that are used to connect the first concentric conductive loops 210 and the second concentric conductive loops 220 in series. More particularly, the connection traces 230 couple the plurality of first concentric conductive loops 210 in series with one another and can couple the plurality of second concentric conductive loops 220 in series with one another. The connection traces 230 can also include a connection trace 235 that couples the outermost first concentric conductive loop 212 with the outermost second concentric conductive loop 214 in series.

As shown in more detail in FIG. 7, the connection traces 230 can be arranged such that fields emanating from the connection traces oppose each other. More particularly, the connection traces 230 can be radially aligned such that a current flow of one of the plurality of connection traces located in the first plane is opposite to a current flow of one of the plurality of connection traces located in the second plane. For instance, referring to FIG. 7, connection trace 232 arranged in the first plane can be nearly radially aligned with connection trace 234 in the second plane. A current flowing in connection trace 232 can be opposite to the current flowing in connection trace 234 such that fields generated by the connection traces 232 and 234 oppose or cancel each other.

As further illustrated in FIG. 7, each of the plurality of first conductive loops 210 and the second conductive loops 220 can include a gap 240 to facilitate connection of the conductive loops using the connection traces 230. The gap can be in the range of about 0.2 mm to about 0.7 mm, such as about 0.5 mm.

The gaps 240 can be offset from one another to facilitate connection of the plurality of concentric conductive loops 210 and 220 in series. For instance, a gap associated with one of the plurality of first concentric conductive loops 210 can be offset from a gap associated with another of the plurality of first concentric conductive loops 210. Similarly, a gap associated with one of the plurality of second concentric conductive loops 220 can be offset from a gap associated with another of the plurality of second concentric conductive loops 220. A gap associated with one of the first concentric conductive loops 210 can also be offset from a gap associated with one of the plurality of second concentric conductive loops 220. Gaps that are offset may not be along the same axis associated with the coil 200.

The coil 200 of FIGS. 6 and 7 can provide a good approximation of the coil contemplated by the quantitative analytical model for magnetic induction tomography imaging. In this way, coil property measurements using the coil 200 can be used to generate three-dimensional electromagnetic property maps of specimens of interest (e.g. human tissue specimens).

FIG. 8 depicts a process flow diagram of an example method (300) for providing a coil for magnetic induction tomography imaging according to example aspects of the present disclosure. FIG. 8 depicts steps performed in a particular order for purposes of illustration and discussion. Those of ordinary skill in the art, using the disclosures provided herein, will understand that the steps of any of the methods disclosed herein can be modified, omitted, rearranged, adapted, or expanded in various ways without deviating from the scope of the present disclosure.

At (302), a plurality of first concentric conductive loops are arranged in a first plane. For instance, the plurality of first concentric conductive loops 210 of the coil 200 of FIG. 6 are arranged on a first plane of a multilayer printed circuit board. At (304) of FIG. 8, a plurality of second concentric conductive loops are arranged in a second plane. For instance, the plurality of second concentric conductive loops 220 of FIG. 6 are arranged on a second plane of a multilayer printed circuit board.

The first plane and the second plane can be separated by a plane separation distance. The plane separation distance can be selected such that the coil approximates a single plane of concentric conductive loops in the analytical model for magnetic induction tomography disclosed herein. For instance, the plane separation distance can be selected to be in the range of about 0.2 mm to about 0.7 mm.

At (306), the plurality of first concentric conductive loops are coupled in series using a plurality of first connection traces in the first plane. At (308) of FIG. 8, the plurality of second concentric conductive loops are coupled in series using a plurality of second connection traces in the second plane. The connection traces can be radially aligned such that fields generated by the connection traces oppose each other. For instance, the connection traces can be arranged such that the plurality of first connection traces and the plurality of second connection traces are radially aligned to connect the plurality of first concentric conductive loops and the plurality of second concentric conductive loops in series such that a current flow of one of the plurality of first connection traces is opposite a current flow of one of the plurality of second connection traces.

At (308), the method can include coupling a first outermost conductive loop located in the first plane with a second outermost conductive loop in the second plane such that the plurality of first concentric conductive loops and the plurality of second concentric conductive loops are coupled in series. For instance, referring to FIG. 6, first outermost conductive loop 212 can be coupled in series with the second outermost conductive loop 222.

At (310) of FIG. 8, the method can include coupling a first innermost conductive loop to an RF energy source. For instance, referring to FIG. 6, an innermost conductive loop 214 of the plurality of first concentric conductive loops 210 can be coupled to an RF energy source. At (312) of FIG. 8, a second innermost conductive loop can be coupled to a reference node (e.g. a ground node or a common node). For instance, referring to FIG. 6, an innermost conductive loop 224 of the plurality of second concentric conductive loops 220 can be coupled to a reference node.

### Example Circuit for Obtaining Coil Property Measurements

FIG. 9 depicts a diagram of an example circuit 400 that can be used to obtain coil property measurements using the coil 200 of FIGS. 6 and 7. As shown, the circuit 400 of FIG. 9 includes an RF energy source 410 (e.g. an oscillator circuit) configured to energize the coil 200 with RF energy. The RF energy source 410 can be a fixed frequency crystal oscillator configured to apply RF energy at a fixed frequency to the coil 200. The fixed frequency can be, for instance, about 12.5 MHz. In one example embodiment, the RF energy source 410 can be coupled to an innermost concentric conductive loop of the plurality of first concentric conductive loops of the coil 200. The innermost concentric conductive loop of the plurality of second concentric conductive loops of the coil 200 can be coupled to a reference node (e.g. common or ground).

The circuit 400 can include one or more processors 420 to control various aspects of the circuit 400 as well as to process information obtained by the circuit 400 (e.g. information obtained by measurement circuit 430). The one or more processors 420 can include any suitable processing device, such as digital signal processor, microprocessor, microcontroller, integrated circuit or other suitable processing device.

The one or more processors 420 can be configured to control various components of the circuit 400 in order to capture a coil loss measurement using the coil 200. For instance, the one or more processors 420 can control a varactor 415 coupled in parallel with the coil 200 so as to drive the coil 200 to resonance or near resonance when the coil 200 is positioned adjacent a specimen for a coil property measurement. The one or more processors 420 can also control the measurement circuit 430 to obtain a coil property measurement when the coil 200 is positioned adjacent the specimen.

The measurement circuit 430 can be configured to obtain coil property measurements with the coil 200. The coil property measurements can be indicative of coil losses of the coil 200 resulting from eddy currents induced in the specimen. In one implementation, the measurement circuit 430 can be configured to measure the real part of admittance changes of the coil 200. The real part of admittance changes of the coil 200 can be converted to real part of impedance changes of the coil 200 as the inverse of admittance for purposes of the analytical model.

The admittance of the coil 200 can be measured in a variety of ways. In one embodiment, the measurement circuit 430 measures the admittance using a phase shift measurement circuit 432 and a voltage gain measurement circuit 434. For instance, the measurement circuit 430 can include an AD8302 phase and gain detector from Analog Devices. The phase shift measurement circuit 432 can measure the phase shift between current and voltage associated with the coil 200. The voltage gain measurement circuit 434 can measure the ratio of the voltage across the coil 200 with a voltage of a sense resistor coupled in series with the coil 200. The admittance of the coil 200 can be derived (e.g., by the one or more processors 420) based on the phase and gain of the coil 200 as obtained by the measurement circuit 430.

Once the coil property measurements have been obtained, the one or more processors 420 can store the coil property measurements, for instance, in a memory device. The one or more processors 420 can also communicate the coil property measurements to one or more remote devices for processing to generate a three-dimensional electromagnetic property map of the specimen using communication device 440. Communication device 440 can include any suitable interface or device for communicating information to a remote device over wired or wireless connections and/or networks.

### Example Methods for Magnetic Induction Tomography Imaging

FIG. 10 depicts a process flow diagram of an example method (500) for magnetic induction tomography imaging according to example aspects of the present disclosure. The method (500) can be implemented by one or more computing devices, such as one or more computing devices of the map generation system 140 depicted in FIG. 1. In addition, FIG. 10 depicts steps performed in a particular order for purposes of illustration and discussion. Those of ordinary skill in the art, using the disclosures provided herein, will understand that the steps of any of the methods disclosed herein can be modified, omitted, rearranged, adapted, or expanded in various ways without deviating from the scope of the present disclosure.

At (502), the method can include accessing a plurality of coil property measurements obtained using a hand held device at a plurality of different discrete locations relative to the specimen. For instance, the plurality of coil property measurements can be accessed from a memory device or can be received from a coil device having a single coil configured for obtaining the coil property measurements. The coil property measurements can be coil loss measurements captured by a single coil when the single coil is energized with RF energy and placed adjacent a specimen at one of the plurality of discrete locations.

In one implementation, the single coil can include a plurality of concentric conductive loops. For instance, the single coil can have a plurality of first concentric conductive loops arranged in a first plane and a plurality of second concentric conductive loops arranged in a second plane. The plurality of concentric conductive loops can be connected using connection traces arranged so as to have a reduced impact on the field created by the coil. For example, the single coil can have the coil geometry of the coil 200 depicted in FIGS. 6 and 7.

The coil property measurements can be obtained at a plurality of discrete positions relative to the specimen. Each coil property measurement can be taken at a different discrete position relative to the specimen. A greater number of coil property measurements can lead to increased accuracy in generating a three-dimensional electromagnetic property map from the coil property measurements.

In a particular embodiment, the coil property measurements can include a plurality of different data sets of coil property measurements. Each of the data sets can be built by conducting a plurality of coil property measurements using a single coil. The single coil can be different for each data set. For instance, each data set can be associated with a single coil having a different overall size and/or outer diameter, relative to any of the other single coils associated with the other data sets. The data sets can be obtained at different times. The data sets can be collectively processed according to example aspects of the present disclosure to generate a three-dimensional map of an electrical property distribution of the specimen as discussed below.

At (504) of FIG. 10, the method includes associating position data with each of the plurality of coil property measurements. The position data for each coil property measurement can be indicative of the position and/or orientation of the single coil relative to the specimen when the coil property measurement was performed. The position data can be associated with each coil property measurement, for instance, in a memory device of a computing system.

The position data can be obtained in a variety of ways. In one implementation, the position data can be obtained for each measurement from data associated with a positioning system configured to determine a position and/or orientation of the hand held device as the hand held device is used to obtain measurements. In addition, signals from one or more sensors (e.g. one or more motion sensors and one or more depth sensors) associated with the hand held device can be also used to determine the position data for a coil property measurement.

At (506), the method includes accessing an analytical model defining a relationship between coil property measurements obtained by the single coil and an electromagnetic property of the specimen. For instance, the analytical model can be accessed, for instance, from a memory device. In one particular implementation, the analytical model correlates a change in an impedance of a single coil having a plurality of concentric conductive loops with a conductivity distribution of the specimen. More particularly, the analytical model can correlate the change in impedance of a single coil with a variety of parameters. The parameters can include the conductivity distribution of the specimen, the position and orientation associated with each coil loss measurement, and the geometry of the coil (e.g., the radius of each of the concentric conductive loops). Details concerning an example quantitative model were provided in the discussion of the example quantitative analytical model for a single coil discussed above.

At (508), the method includes evaluating the analytical model based on the plurality of coil property measurements and associated position data. More particularly, an inversion can be performed using the model to determine a conductivity distribution that most closely leads to the plurality of obtained coil property measurements. In one example aspect, the inversion can be performed by discretizing the specimen into a finite element mesh. The finite element mesh can include a plurality of polygonal elements, such as tetrahedral elements. The shape and resolution of the finite element mesh can be tailored to the specimen being analyzed. As a matter of practicality, the coil location data can be used to avoid meshing those regions of space visited by the coil, improving efficiency. Once the finite element mesh has been generated for the specimen, a conductivity distribution for the finite element mesh can be computed using a non-linear or constrained least squares solver.

More particularly, a plurality of candidate electromagnetic property distributions can be computed for the finite element mesh. Each of these candidate electromagnetic property distributions can be evaluated using a cost or objective function, such as the root mean square error. The cost or objective function can assign a cost to each candidate electromagnetic property distribution based at least in part on the difference between the obtained coil property measurements and theoretical coil property measurements using the model. The candidate electromagnetic property distribution with the lowest cost can be selected as the electromagnetic property distribution for the specimen. Those of ordinary skill in the art, using the disclosures provided herein, will understand that other suitable techniques can be used to determine an electromagnetic property distribution using the analytical model without deviating from the scope of the present disclosure.

At (510), a three-dimensional electromagnetic property map can be generated based on the electromagnetic property distribution identified using the inversion algorithm. The three-dimensional property map can provide an electromagnetic property distribution (e.g., a conductivity distribution) for a plurality of three-dimensional points associated with the specimen. Two-dimensional views along cross-sections of the three-dimensional electromagnetic property map can then be captured and presented, for instance, on a display device. Three-dimensional views of the electromagnetic property map can also be generated, rotated, and presented, for instance, on a display device.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A magnetic induction tomography imaging system, comprising:
a hand held magnetic induction tomography device, the hand held magnetic induction tomography device comprising a housing and at least one sensing unit, each sensing unit comprising a single coil, the hand held magnetic induction tomography device configured to obtain a coil measurement associated with the single coil when the single coil is energized with RF energy and placed adjacent to a specimen;
a positioning system configured to determine a position of the hand held magnetic induction tomography device associated with each coil measurement; and
a map generation system configured to generate an electromagnetic property map of at least a portion of the specimen based at least in part on the coil measurement associated with the single coil.

2. The magnetic induction tomography imaging system of claim 1, wherein the housing has a form factor to facilitate holding by hand when obtaining the coil measurement.

3. The magnetic induction tomography imaging system of claim 2, wherein the housing has a form factor such that the location of a hand grasping the housing when in operation is separated a threshold distance from the single coil.

4. The magnetic induction tomography imaging system of claim 3, wherein the threshold distance is in the range of about 0.5 inches to about 4 inches.

5. The magnetic induction tomography imaging system of claim 1, wherein the hand held device comprises one or more electrical components or mechanical components disposed in the housing.

6. The magnetic induction tomography imaging system of claim 5, wherein the one or more electrical components or mechanical components comprise one or more of the map generation system, an RF energy source, one or more processors, one or more memory devices, one or more positioning devices, one or more communications devices, or a measurement circuit.

7. The magnetic induction tomography imaging system of claim 5, wherein the one or more electrical components or mechanical components are separated from the single coil of the at least one sensing unit by a threshold distance.

8. The magnetic induction tomography imaging system of claim 7, wherein the threshold distance is in the range of about 0.5 inches to about 4 inches.

9. The magnetic induction tomography imaging system of claim 5, wherein the hand held magnetic induction tomography device further comprises shielding separating the single coil of the at least one sensing unit from the one or more electrical components or mechanical components.

10. The magnetic induction tomography imaging system of claim 1, wherein the hand held magnetic induction tomography device is in communication with the map generation system over a wired or wireless communications interface.

11. The magnetic induction tomography imaging system of claim 1, wherein the positioning system comprises one or more of an electromagnetic positioning system, optical sensing system, or an acoustic positioning system.

12. The magnetic induction tomography imaging system of claim 1, wherein the positioning system is configured to determine a position and an orientation of the hand held device associated with each coil measurement.

13. The magnetic induction tomography imaging system of claim 1, wherein the positioning system comprises one or more motion sensors or depth sensors on the hand held magnetic induction tomography device.

14. A hand held magnetic induction tomography device comprising:
a housing having a form factor to facilitate holding by hand;
at least one sensing unit, each sensing unit comprising a single coil;
one or more electrical components separated from the at least one sensing unit a sufficient distance to reduce electromagnetic interference between the one or more electrical components and the at least one sensing unit;
wherein the hand held magnetic induction tomography device is configured to obtain a coil measurement from the sensing unit when the single coil is energized with RF energy and placed adjacent to a specimen.

15. The hand held magnetic induction tomography device of claim 14, wherein the one or more electrical components comprise one or more of the map generation system, an RF energy source, one or more processors, one or more memory devices, one or more positioning devices, one or more communications devices, or a measurement circuit.

16. The hand magnetic induction tomography device of claim 15, wherein the one or more electrical components are separated from the single coil of the at least one sensing unit by a threshold distance.

17. The hand magnetic induction tomography device of claim 15, wherein the hand held magnetic induction tomography device further comprises shielding separating the single coil of the at least one sensing unit from the one or more electrical components.

18. A method for magnetic induction tomography imaging, the method comprising:
accessing a plurality of coil property measurements obtained for a specimen using a single coil of a hand held magnetic induction tomography device, the single coil coupled to and energized with radio frequency energy from an RF energy source, each of the plurality of coil property measurements obtained with the single coil at one of a plurality of discrete locations relative to a specimen, each coil property measurement comprising a detected coil property of the single coil coupled to and energized with radiofrequency energy from an RF energy source; each of the plurality of coil property measurements obtained with the single coil at one of a plurality of discrete locations relative to a specimen;
associating coil position data with each of the plurality of coil property measurements, the coil position data indicative of the position and orientation of the single coil relative to the specimen for each coil measurement, the coil position data obtained using a positioning system configured to determine a position of the hand held magnetic induction tomography device;
accessing a model defining a relationship between coil property measurements obtained by the single coil and an electromagnetic property of the specimen; and
generating a three-dimensional electromagnetic property map of the specimen using the model based at least in part on the plurality of coil property measurements and the coil position data associated with each coil measurement.

19. The method of claim 18, wherein the hand held device comprises a housing having a form factor to facilitate holding by hand when obtaining the coil measurement.

20. The method of claim 19, wherein the housing has a form factor such that the location of a hand grasping the housing when in operation is separated a threshold distance from the single coil, the threshold distance being in the range of about 0.5 inches to about 4 inches.
